(19) United States
(12) Patent Application Publication
(10) Pub. No.: US 2022/0192768 A1
(43) Pub. Date: Jun. 23, 2022

(54) ROBOTIC SURGERY SYSTEM AND METHOD FOR CONTROLLING SAME

(71) Applicant: avateramedical GmbH, Jena (DE)

(72) Inventors: Tobias GASSNER, Kronau (DE); Ricardo A. TAURO, Karlsruhe (DE); Tobias LUKSCH, Rülzheim (DE); Lars SPITTEL, Ilmenau (DE)

(21) Appl. No.: 17/554,289

(22) Filed: Dec. 17, 2021

(30) Foreign Application Priority Data
Dec. 22, 2020 (DE) 102020134626.3

(51) Int. Cl. A61B 34/37; A61B 34/00

(52) U.S. Cl. CPC A61B 34/37; A61B 2034/2059; A61B 34/75; A61B 34/74

(57) ABSTRACT

A method for controlling, by means of an input unit, at least one manipulator arm of a robotic surgery system and an instrument held by the at least one manipulator arm. The input unit features a multi-linkage kinematic chain with active and/or passive joints and an open end. The open end is configured as a handpiece with at least one first passive joint. An operator controls movements of the instrument via moving the handpiece. The first passive joint features a first position sensor creating position signals which are used to control the movements of the instrument. A first measured value is calculated from a chronological succession of position signals of the first position sensor, and compared with a specified first threshold value. Control of the movement of the instrument is interrupted when the first measured value is smaller than the first threshold value.

### PRIORITY CLAIM

The present application claims priority to German Patent Application No. 10 2020 134 626.3, filed on Dec. 22, 2020, which said application is incorporated by reference in its entirety herein.

### FIELD OF THE INVENTION

The invention relates to a robotic surgery system with at least one manipulator arm and a method for controlling, by means of an input unit, the at least one manipulator arm and an instrument held by the at least one manipulator arm. The invention further relates to the problem of recognizing, as precisely as possible, whether an operator—as a rule, a surgeon—is operating the surgery system, or preparing it for operation, or stops operating the surgery system. Recognizing this as precisely as possible helps making the system more proof against operating errors which, in the worst case, may risk the life of a patient receiving treatment.

### BACKGROUND OF THE INVENTION

As a rule, robotic surgery systems comprise a console for handling the manipulator arm. As a rule, the console is separated from, and movable relatively to, the rest of the surgery system including one or several manipulator arms. The manipulator arm is configured as a kinematic chain with a multiplicity of joints, and with an instrument being arranged in an instrument holder at the end of the chain. Part of the joints of the manipulator arm are used for prepositioning and are fixed prior to surgery, whereas the remaining joints can be moved during surgery by means of the input unit. This part of the joints, including the instrument holder and possibly the instrument, are imaged in the console as an input unit with a multi-linkage kinematic chain having active and/or passive joints. The input unit further comprises an open end connected to the multi-linkage kinematic chain. An active joint is understood to be a motor-driven joint. In case of an active pivot joint, for example, the rotation is motor-driven, with the motor being triggered by a control unit, as a rule. Passive joints, on the other hand, are not motor-driven but freely movable, the motion either being effected by hand or in combination with other, active joints.

The operation of the input unit is adjusted for the easiest possible movability; this is achieved by controlled weight compensation, which is the sole movement assistance in the input unit if the latter is coupled with a manipulator arm. Usually, a console is equipped with two input units, so that an operator can handle two manipulator arms at a time, as is the case for a surgeon standing at the operating table. For controlling the movements of the instrument, the operator uses a handpiece. This requires the provision of a mechanism that ensures that the instrument is moved only if it is controlled by the operator through the input unit. If the operator is not operating the input unit, inadvertent movements must be prevented from being transmitted to the instrument. Therefore, it is necessary to detect whether or not the operator is ready to operate the instrument through the input unit. Various approaches to solving this problem are already known in prior art.

US 2011/118748 A1 discloses a system that automatically detects the presence of a hand near the handpiece. Means proposed are, e.g., capacitive switches, pressure switches, or switches operating on an infrared basis. In WO 2018/162921 A1, a switch at the input unit allows the operator to switch active control on or off.

US 2015/0100066 A1 also specifies sensors that check the presence of a hand on the handpiece. For example, a lever-button mechanism is described. Controlling the movement of the instrument is allowed only if the force exerted on the handpiece exceeds a defined minimum level, for which purpose another sensor is required that senses the force exerted.

The handpiece described in US 2018/0168758 A1 is equipped with a multiplicity of distance sensors to detect whether or not the operator's hand is gripping the handpiece. For examples of such sensors, this document generally refers to optical or capacitive sensors. A similar approach is followed in WO 2019/217882 A1; examples mentioned here include optical and pressure sensors.

US 2003/0060927 A1 follows a different approach. Here, the driven joints are employed to determine whether or not an operator touches the handpiece. For that purpose, the motor causes the active joints to oscillate with very low amplitudes. If an operator grips the handpiece, the oscillation pattern will change. The oscillation pattern can be detected by means of a joint sensor, e.g., a potentiometer.

### SUMMARY OF THE INVENTION

Sometimes, just if one aims at ease of motion of the input unit, the mechanisms described in prior art are too imprecise, or require several special sensors that are not absolutely imperative for moving the input unit, or need active joints for generating oscillations, which might make themselves felt annoyingly.

Therefore, the problem of the invention is to develop a method for controlling at least one manipulator arm of a robotic surgery system that, for obtaining ease of motion, makes it possible to detect, without additional elements or with the least possible number of them, whether or not a hand is resting on the input unit. A further problem of the invention is the development of an appropriate robotic surgery system.

This problem is solved by a method for controlling at least one manipulator arm of a robotic surgery system and an instrument held by the at least one manipulator arm, with the control being effected by means of an input unit featuring a multi-linkage kinematic chain with active and/or passive joints and an open end. The open end of the input unit is configured as a handpiece with at least one first passive joint. An operator controls the motions of the instrument by way of moving the handpiece. The first passive joint is equipped with a first position sensor, and position signals of the first position sensor are employed to control the movements of the instrument. From a chronological succession of the position signals of the first position sensor, a first measured value is calculated and compared with at least one specified first threshold value. In case the first measured value is smaller than the first threshold value, control of the instrument movement is interrupted.

The first position sensor is already provided for controlling the instrument movement, so that no additional element needs to be installed. It is only necessary to adapt the control accordingly so that the chronological succession of the position signals of the first position sensor can be analyzed. In the simplest case, a position difference is calculated from the chronological succession of the position signals. For that purpose, at least two position signals following each other in time are required. If this difference is smaller than a specified threshold value, which also corresponds to a position difference, control of the instrument movement is interrupted, i.e., an active change of the instrument's position is stopped. For the kind of the analysis, there are various methods basically known to persons skilled in the art. The precision of the analysis, for example, can be improved by using not only two, but more than two chronologically successive position signals. In that case, what is looked at is not the mere difference of the position signals, but their standard deviations, wherein position signals farther back are assigned less—e.g., exponentially less—weight so that the influence exerted by these signals on the value of the standard deviation is the slighter the farther back in time these signals originated.

In a particularly preferred embodiment, the first threshold value is specified in such a way that the presence of a physiological tremor and, thus, of an involuntary movement is excluded if the first measured value falls short of the first threshold value. Physiological tremor is an involuntary high-frequency myokinesis with an amplitude that is very low at or below room temperature, and that is superimposed to every other muscle movement. While physiological tremor is present also in case of short movements with a large position change, it is superimposed by the movement and, thus, hardly perceptible. As a rule, it becomes perceptible only in particularly critical movements near standstill; the amplitudes increase in the cold. Specifying the first threshold value is based on room temperature, though. Therefore, if the first threshold value is geared to the physiological tremor or to the amplitudes/deflections or position changes caused by physiological tremor, it can be determined with especially high accuracy whether the operator's hand is still at the handpiece. Thus, if the first measured value is below the first threshold value, the control system interprets this as the operator having let go of the handpiece.

Subsequently, it is of advantage to fix at least the active joints in their current positions, i.e. the joints and the links of the input unit can no longer be moved by the operator if he/she, for example, inadvertently attempts to move one of the links without gripping the handpiece. The fixation of the active joints can be effected via the motor drive and appropriate arresting means. The passive joints, too, can be fixed by appropriate fixation mechanisms; otherwise, the parts of the joints in the non-fixed state are freely movable relative to each other. Preferably, fixing the joints is effected by a retractive force. With the active joints, this retractive force is implemented by means of the control system, which ensures that the links connecting the joints behave like bodies resiliently held in their respective positions. In case the control system has, for a short time, erroneously detected a release of the handpiece, the operator will not be impeded; i.e., once the minimal position change has been detected, control of the joint positions by the operator is reactivated.

In a particularly preferred embodiment of this version, control of the instrument movement is interrupted only at the end of a specified period. If the analysis of measured values yields the result that the first measured value is smaller than the first threshold value, a timer is started, and only if, within the specified period after the start of the timer, the first measured value permanently remains smaller than the first threshold value, control is interrupted after the specified period—as a rule, not more than five seconds—has expired and, as a consequence, coupling between the input unit and the manipulator arm is cancelled so that no involuntary movement of the instrument or the instrument's tip can take place any more.

In another preferred embodiment of the method, the first measured value is, in addition, compared with a second threshold value that is specified in such a way that exceeding the second threshold value by the first measured value conforms to the presence of a movement deliberately initiated by the operator, with the second threshold value being greater than the first threshold value. The second threshold value can, for example, be specified in such a way as, with the joints fixed, to conform to a possible deflection effected by an operator, against the effect of the retractive force generated by the control system via the driven joints, for a position change markedly exceeding the physiological tremor in order to indicate that the operator wants to handle the instrument or the manipulator arm. If the first measured value is greater than the second—and, thus, also the first—threshold value, the instrument is enabled to be moved, i.e., control by the operator is allowed, and a fixation of the joints possibly effected before is abolished. The second threshold value may be specified, e.g., as a minimum amplitude of a defined activation movement. If only the first threshold value is defined, enabling is effected already if the first measured value is greater than the first threshold value, e.g., in case of sufficiently large deflections against the retractive force as described above.

For even higher safety, it is expedient to enable movement only if additional, pre-defined criteria confirm that the operator has taken up an operating position. Such a criterion may consist, e.g., in a check whether the operator's head is in an operating position, i.e., whether it is visible on a suitable screen showing the patient's detail to be operated on.

Frequently, controlling the instrument requires more than one degree of freedom realizable by means of a simple joint. A pair of forceps or scissors, e.g., may be opened or closed as well as rotated about a longitudinal axis. In another preferred embodiment, therefore, the handpiece additionally features a second passive joint equipped with a second position sensor. Analogously to processing the position signals of the first position sensor, a second measured value is calculated from a chronological succession of position signals of the second position sensor and compared with the first threshold value. If a second threshold value has been defined, too, as described above, the second measured value is also compared with the second threshold value. The criteria for interrupting control of the instrument's movement and enabling that movement are applied analogously, with interruption of control being effected only if both the first and the second measured value are smaller than first threshold value. For cancelling the interruption of control, on the other hand, it is sufficient if either the first measured value or the second measured value exceeds —, i.e., is greater than—the first threshold value or the second threshold value if this has been defined. Thus, the first or second threshold value, respectively, needs to be exceeded at only one of the joints in order to return control to the operator. This can, for example, be the case if the rotary position of a pair of forceps needs to remain stable, whereas it is intended to be opened or closed.

As an alternative or additional feature, the control system is required to receive at least one additional activation signal before it is enabled, with the activation signal being independent of a movement. This may be implemented, e.g., by a pedal switch or a separate button that needs to be depressed. This will enhance the safety of controlling the surgery system.

Moreover, it is possible to assign different first and/or second threshold values to the first position sensor and the second position sensor, which is an advantage if, for example, the first passive joint and the second passive joint implement different types of joints. For example, the first passive joint may be a pivot joint by which an instrument is rotated about its longitudinal axis, whereas the second passive joint may be a sliding joint or a pivot joint by which the instrument, e.g., a pair of scissors, is opened or closed.

A robotic surgery system that solves the problem outlined above comprises at least one manipulator arm and an instrument carried by the manipulator arm. The robotic surgery system further comprises an input unit for handling the at least one manipulator arm, with the input unit featuring a multi-linkage kinematic chain with active and/or passive joints and an open end. Moreover, the robotic surgery system comprises a control unit that converts movements of the input unit into movements of the manipulator arm and the instrument. The open end of the input unit is configured as a handpiece for controlling the movements of the instrument by an operator. It comprises at least a first, but, as a rule, at least a first and a second passive joint, because but few instruments are conceived to have a single degree of freedom that is adjustable by a joint. The first passive joint is equipped with a first sensor unit comprising a first position sensor. The second passive joint is equipped with a second sensor unit comprising a second position sensor. The first sensor unit is adapted to successively transmit first position signals of the first position sensor to the control unit. Analogously, the second sensor unit is adapted to successively transmit second position signals of the second position sensor to the control unit. The control unit is adapted to calculate a first measured value from the chronological succession of first position signals, and a second measured value from the chronological succession of second position signals. The control unit is furthermore adapted to interrupt control of the movement of the instrument if the first measured value and, if provided, the second measured value are smaller than a specified first threshold value.

To the first and the second measured value and the first threshold, the statements made about the method apply analogously. In particular, the first threshold value is specified in such a way that, in case the first and second measured values fall short of the first threshold value, a physiological tremor and, thus, an involuntary movement are excluded. Also, a second threshold value may be defined that is greater than the first threshold value and specified in such a way that exceeding the second threshold value by the first or second measured value corresponds to the presence of a movement deliberately initiated by the operator. Further, the first and the second threshold value may differ for the two position sensors. If no second threshold value is specified, exceeding the first threshold value by the first or second measured value alone corresponds to the presence of a movement deliberately initiated by the operator.

Preferably, the robotic surgery system further comprises fixation means, which fix at least the active joints in their current positions in case the first measured value and the second measured value fall short of the first threshold. The said means may be, e.g., a special mechanical element or structure that prevents the links from moving relatively to each other at a joint. Alternatively, the said means may be a special control element, especially implemented by software, that interacts with an arresting motor, in which case the joints are fixed in their current positions by the control system, preferably by the action of a retractive force. Provided that the joints are fixed, an optional timing circuit will, after a specified period of time has expired, cancel the coupling between a console—in which the input unit and, as a rule, the control unit are integrated—and the at least one manipulator arm, so that movements of the input unit are no longer transmitted to the manipulator arm.

It is understood that the features mentioned above and those to be explained below are applicable not only in the combinations stated but also in other combinations or as stand-alone features without leaving the scope of the present invention.

Below, the invention will be explained in more detail on the basis of exemplary embodiments with reference to the accompanying drawings, which also disclose features essential to the invention. These exemplary embodiments merely serve the purpose of illustration and must not be interpreted as restrictive. For example, a description of an exemplary embodiment featuring a multiplicity of elements or components must not be interpreted in the sense that all these elements or components are needed for an implementation. Rather, other exemplary embodiments may also contain alternative elements and components, fewer elements or components, or additional elements or components. Elements or components of different exemplary embodiments may be combined with each other unless stated otherwise. Modifications and variations described for one of these exemplary embodiments may also be applicable to other exemplary embodiments.

### BRIEF DESCRIPTION OF THE DRAWINGS

To avoid redundancies, similar or mutually corresponding elements in different drawings are designated by the same reference numbers and explained a single time only. Of the drawings,

FIG. 1 shows a general overview of a robotic surgery system,

FIG. 2 is a more detailed drawing of a console of the surgery system,

FIG. 3 shows the details of an input unit as part of the console,

FIG. 4 shows an embodiment of a handpiece, and

FIG. 5 is a typical flow chart of the control processes of the surgery system.

### DETAILED DESCRIPTION

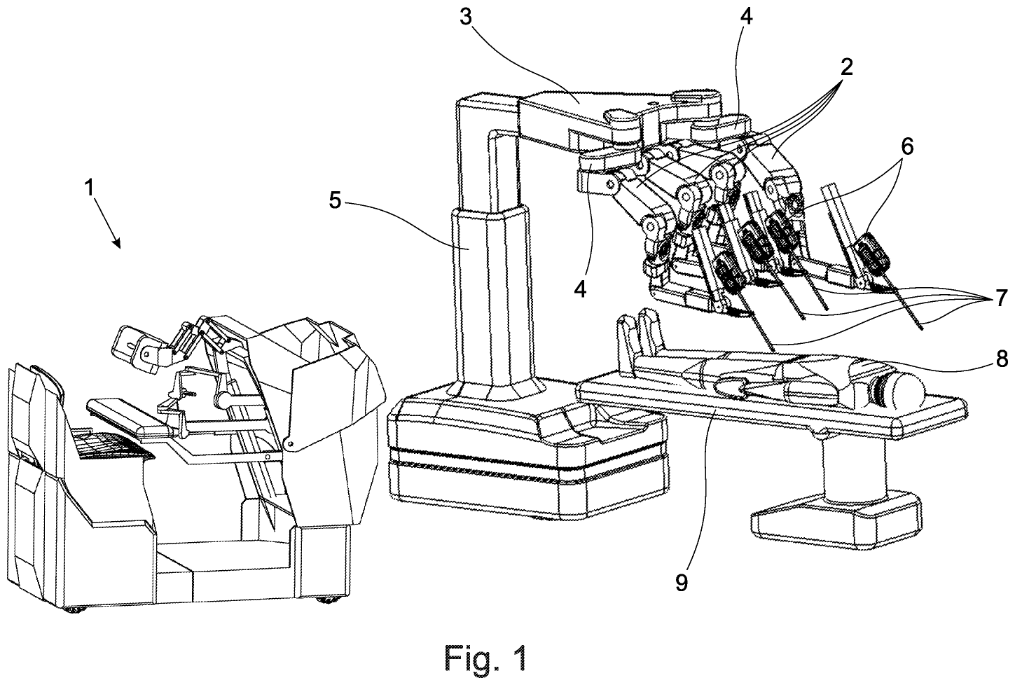

First of all, FIG. 1 shows a general overview of a robotic surgery system. Shown on the left is a console 1, from which an operator handles one or several manipulator arms 2 that are fastened to a cross member 3 via coupling elements 4. The cross member 3 is fastened to a supporting column 5, the height of which is adjustable. The manipulator arms 2 are configured as kinematic chains, with one of their respective ends being fastened to the cross member 3. Movably coupled to the respective other, open ends are instrument holders 6, which hold instruments 7. This drawing only illustrates the principle; in reality, the instruments 7 will vary, including, e.g., scissors, forceps, hooks, and/or endoscopes. From the console 1, the operator, by handling the bottom parts of the manipulator arms 2 with the instruments 7, performs surgery on a patient 8 recumbent on an operating table 9.

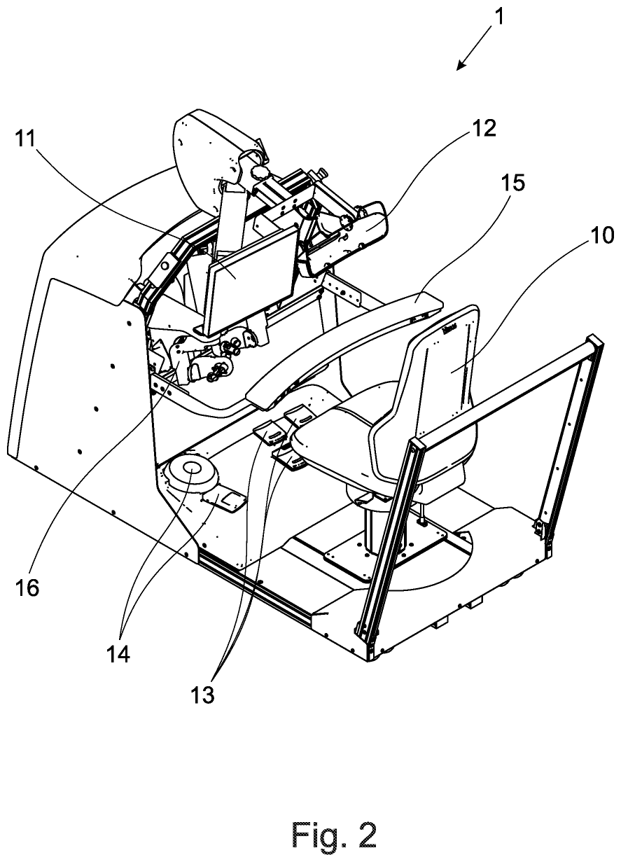

The movable console 1 is explained below in more detail and with reference to FIG. 2. Integrated in it is a control unit, which is not visible from outside and therefore not shown in FIG. 2. The operator sits down on an adjustable chair 10. An overview screen 11 provides him/her with an overview of the operating site. Next to it on the right there is an operator screen 12, on which the operator sees a cutout of the operating site showing the detail currently operated on. In addition, the operator screen 12 comprises means for detecting whether the operator is in position and looking at the operator screen 12. The means for detecting this may comprise, e.g., a detector, an eye tracking device or other sensors that detect whether a head is visible on the display, such as optical proximity sensors with distance measurement, or a plain photoelectric beam. For handling the manipulator arms 2, various pedal switches 13, 14 are provided, which can also initiate a change-over of handling between two manipulator arms 2. During surgery, the operator can rest his elbows or forearms on an arm rest 15.

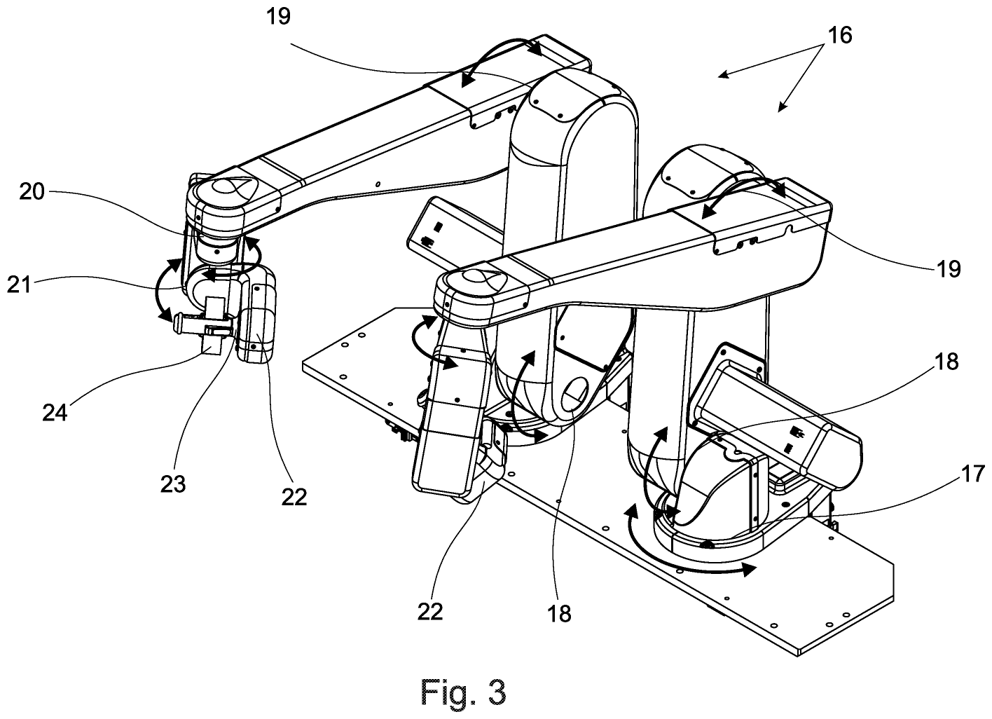

The control or handling, respectively, of the bottom parts of the manipulator arms 2 with the instruments 7 is effected via an input unit 16, which is explained in more detail with reference to FIG. 3. This drawing shows two input units 16 for handling two manipulator arms 2. Since the manipulator arms 2 are configured as kinematic chains, it is expedient if the input units 16 for handling a manipulator arm 2 are each equipped with a multi-linkage kinematic chain with active and/or passive joints, and with an open end. The movements of the input units 16 are converted into movements of the manipulator arm 2 and of the instrument 7 by the control unit. The input units 16 shown here are configured as kinematic chains, with altogether five joints 17, 18, 19, 20 each, the positions of which are but roughly indicated in FIG. 3. The said joints are pivot joints with one degree of freedom each; the rotation directions are indicated by the double arrows next to each joint. In the example shown, the joints 17-21 are active joints, i.e., they are motor-assisted. The three joints 17-19, if coupled with a manipulator arm 2, control the spatial position of the instrument 7.

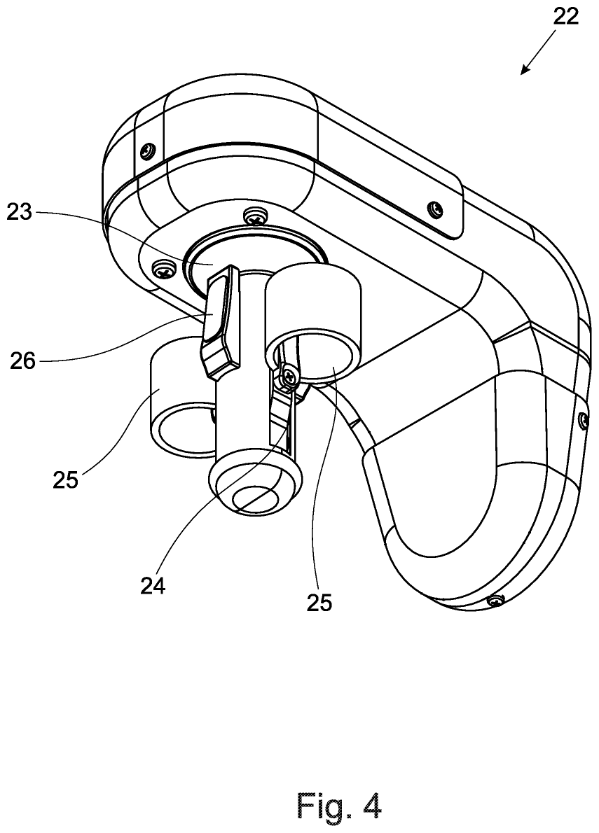

The open end of each input unit 16 is configured as a handpiece 22 for controlling movements of the instrument 7 by the operator. The handpiece 22 selected here as an example is shown in detail in FIG. 4. It is designed for use with an instrument 7 equipped with a jaw part. Such instruments are, for example, forceps or scissors. Below, the function is illustrated for a pair of forceps as an example. The handpiece 22 comprises a first passive joint 23 and a second passive joint 24. The joints 20-24 are intended to control the movement of the instrument tip, once the spatial position has been set and fixed. By means of the first passive joint 23, the rotation of the instrument 7 about its longitudinal axis is implemented. The first passive joint 23 is equipped with a first sensor unit—not shown here—inside the handpiece 22, which comprises a first position sensor. The first position sensor detects the rotary position of the first passive joint 23 in a range between 0° and 360°. The second passive joint 24 is equipped with a second sensor unit—not shown here either—inside the handpiece 22, which comprises a second position sensor. The positions for both passive joints 23 and 24 are determined in absolute terms by means of an absolute encoder, so that the angular deflections can be output without comparison with a reference value. The second passive joint 24 is equipped with two finger loops 25, into which the operator can put, e.g., his/her thumb and index or middle finger to open and close the forceps. A suitable mechanism is described, e.g., in DE 10 2017 103 199 A1. The aperture angle—corresponding to a deflection of the finger loops from a rest position—is determined via the second position sensor, which can be integrated in the movement mechanism. The handpiece 22 is further equipped with a finger coupler 26, with which—besides other functions—an activation movement can be performed, which signalizes to the control unit that an operator takes over control of the respective input unit 16. The control unit then releases control as soon as the operator's head appears on the operator screen 12. With the handpiece 22 one can, of course, also control other instruments with a single degree of freedom such as, e.g., endoscopes. Here, with an angled radiation surface, one only needs the capability of rotation about the longitudinal axis by means of the first passive joint 23; a movement of the second passive joint 24, then, will not effect any control of instrument 7.

Now the first sensor unit successively sends first position signals of the first position sensor to the control unit; accordingly, the second sensor unit successively sends second position signals of the second position sensor to the control unit. The control unit determines a first measured value from the chronological succession of the first position signals, and a second measured value from the chronological succession of the second position signals. If the first measured value and the second measured value are smaller than a specified first threshold value, the control unit interrupts the control of the movements of the instrument. The first threshold value is specified to the effect that the first and the second measured value falling short of the first threshold value excludes the presence of a physiological tremor and, thus, of an involuntary movement. This requires that the sensitivities of the first and the second position sensor are high enough to detect a physiological tremor or the deflection amplitudes related thereto. A resolution of the first position sensor of, e.g., 16 bit—corresponding to an angular resolution of 0.005° per increment—has proved to be sufficient. The first threshold value, then, can be set, e.g., at 0.005° as a lower limit, but may also amount to a few increments. Also, the first threshold value for the first position sensor may differ from that for the second position sensor, i.e., each of the position sensors can be assigned its own first threshold value. This is advantageous especially if the joints differ markedly from each other.

For calculating the first measured value and the second measured value there are different possible ways. The simplest way consists in subtracting two successive position signals from each other and checking whether the difference is greater or smaller than the first threshold value. To increase accuracy, however, one can analyze a longer-time succession of position signals. For example, one can successively determine the standard deviations of the positions of the first passive joint and the second passive joint and lay these down as the first or second measured value, respectively. It is particularly advantageous to ascribe less weight to position signals from longer ago and assign a damping factor to them.

In addition, one can specify a second threshold value and also compare the first and the second measured value with it. The second threshold value is specified to the effect that the first and/or the second measured value exceeding the second threshold value corresponds to the presence of a movement deliberately initiated by the operator. Therefore, the second threshold value is markedly greater than the first threshold value. If one of the two measured values exceeds the second threshold value, the control unit can interpret this as an activation of the input unit to take over control by the operator, if, while it has been detected that the operator has released the handpiece, a period until the interruption of control and, thus, the coupling between the input unit and the manipulator arm has not yet expired. In that case, an additional activation signal is not required.

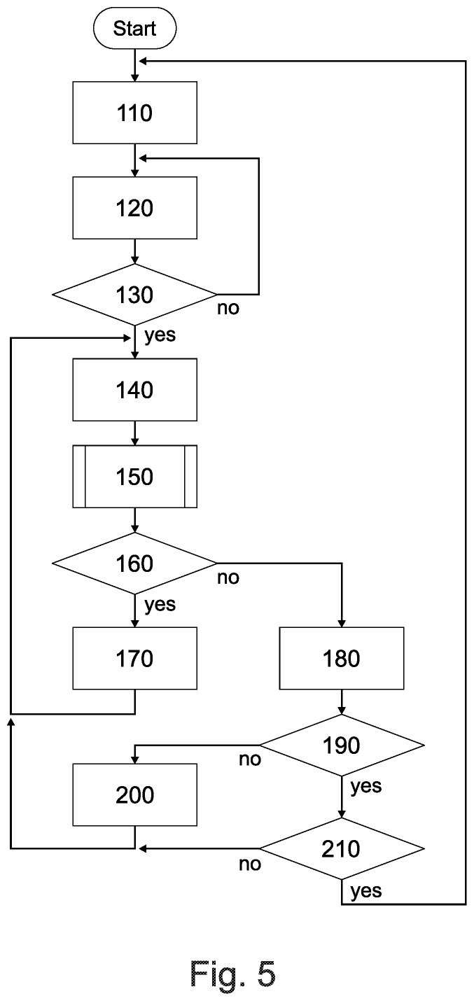

The course of a procedure to operate a robotic surgery system, in which the system detects whether or not an operator controls the input unit, is explained below with the aid of FIG. 5. Upon the system being started, the control unit in a step 110 first blocks the possibility of the operator to control the input unit 16. During a time loop in step 120, the control unit waits for the operator to trigger an activation signal. This activation signal can be triggered, e.g., by the operator pulling the finger coupling 26 away along the axis of the handpiece 22. Alternatively, an activation signal can also be achieved by an increased deflection of the second passive joint 24 by means of the autopodia inserted in the finger loops 25, which supplies a measured value that exceeds the second threshold value. For this activation movement there are two possibilities. If the instrument tip is closed, activation is achieved by moving the finger loops 25 away from each other and then towards each other again, which correspond to an opening. If, on the other hand, the instrument tip is open, activation is achieved by moving the finger loops 25 towards each other, which corresponds to a closing, and then away from each other again. Further, to be enabled to take over control of the input unit 16, the operator needs to appear on the display of the operator screen 12. In a step 130, the control unit checks whether all conditions for control take-over by the operator are satisfied. If this is not the case, the loop in step 120 is passed once again.

If, however, activation is confirmed, control of the input unit 16 will be taken over by the operator. Subsequently, in step 140 the first and the second sensor unit continuously send position signals of the first and the second position sensor to the control unit. In step 150, the control unit computes a first measured value from the chronological succession of the position signals of the first position sensor, and a second measured value from the chronological succession of position signals of the second position sensor, and compares these measured values with the first threshold value. This is specified to the effect that, with both the first and the second measured value falling short of the first threshold value, excludes an involuntary movement of the elements of the handpiece 22, caused by a physiological tremor of the operator.

Step 160 queries whether the operator continues controlling the input unit 16. This is the case if at least one of the two measured values is greater than the first threshold value. In this case, step 170 is carried out, and the operator maintains control of the input unit, i.e. the active and/or passive joints will not get fixed, and the data of the two position sensors will continue to be read out. However, the operator will not control the input unit 16 if both measured values are smaller than the first threshold value. In that case, step 180 will be carried out, and the control unit takes over control of at least the active joints of input unit 16, and the active joints—here, the joints 17-22—are fixed in their current positions under the action of a retractive force; this is implemented by the control unit and the motor drives of the joints. In this so-called impedance control, the joints, or the links connecting the joints, behave like solids held in their positions by springs, so that minimum position changes are possible, also in order to enable reactivation.

Step 190, then, queries whether a timer has been started yet. If this is not the case, the timer is started in step 200, and the position signals of the two position sensors continue to be analyzed. Should this result in at least one of the two measured values to be greater than the first threshold value, which means that the operator has not yet released the handpiece 22, the timer is stopped, and full control of the input unit 16 will be returned to the operator. Due to the action of the retractive force, the input unit 16 will, in this case, behave in a way that does not affect the operator.

In case the timer has already been started, step 190 is followed by step 210, which queries, whether a specified period—as a rule, between three and five seconds, although shorter or longer periods can be specified as well—has expired. If this is the case, control is interrupted between the input unit 16 and the manipulator arms 2 in addition to the fixation of the joints of the input unit 16, and the procedure continues with step 110. If the operator wants to continue operation, he/she has to carry out an activation movement again.

By the method described above and with a robotic surgery system implementing that method, it is possible to realize, essentially without additional technical components and with high accuracy, a control system that ascertains whether an operator is ready or not to operate the input unit of the surgery system. For this purpose, the system uses data—the position signals of position sensors—that accumulate anyway in operating the robotic surgery system and, in particular, the instrument; these data are subjected to a supplementary analysis, possibly by computation.

### LIST OF REFERENCE NUMBERS

1 console
2 manipulator arm
3 cross member
4 coupling assembly
5 supporting column
6 instrument holder
7 instrument
8 patient
9 operating table
10 adjustable chair
11 overview screen
12 operator screen
13 pedal switches
14 pedal switches
15 hand rest
16 input unit
17 joint
18 joint
19 joints
20 joint
21 joint
22 handpiece
23 first passive joint
24 second passive joint
25 finger loop
26 finger coupling

1. A method for controlling at least one manipulator arm of a robotic surgery system and an instrument held by the at least one manipulator arm, comprising:
controlling a handpiece of an input unit that is equipped with a multi-linkage kinematic chain provided with active and/or passive joints and an open end, wherein the open end is configured as the handpiece, the handpiece including at least one first passive joint, by the movement of which an operator controls movements of the instrument wherein the first passive joint is provided with a first position sensor, and position signals of the first position sensor are used to control the movement of the instrument,
calculating a first measured value from a chronological succession of the position signals of the first position sensor,
comparing the first measured value with at least one specified first threshold value, and
interrupting control of the movement of the when the first measured value is smaller than the first threshold value.

2. The method as claimed in claim 1, wherein the first threshold value is specified to the effect that, when the first measured value is smaller than the first threshold value, the presence of a physiological tremor and, thus, an involuntary movement, are ruled out.

3. The method as claimed in claim 1, wherein, when control of the movement of the instrument is interrupted, at least the active joints get fixed in their current positions.

4. The method as claimed in claim 3, wherein at least the active joints are fixed under the action of a retractive force.

5. The method as claimed in claim 1, wherein control is interrupted only after expiry of a specified period.

6. The method as claimed in claim 1, wherein the interruption of control is cancelled and movement of the instrument is enabled if the first measured value is greater than the first threshold value and/or if the control unit receives an activation signal that is independent of the movement.

7. The method as claimed in claim 1, wherein the first measured value is, in addition, compared with a second threshold value, and the second threshold value is specified to the effect that the first measured value being in excess of the second threshold value corresponds to the presence of a movement deliberately initiated by the operator, with the second threshold value being greater than the first threshold value.

8. The method as claimed in claim 7, wherein the interruption of control is cancelled and movement of the instrument is enabled if the first measured value is greater than the second threshold value.

9. The method as claimed in claim 8, wherein the said enabling takes place only if it is confirmed on the basis of additional, predefined criteria that the operator has taken up a working position.

10. The method as claimed in claim 6, wherein the said enabling takes place only if it is confirmed on the basis of additional, predefined criteria that the operator has taken up a working position.

11. The method as claimed in claim 1, wherein the handpiece is, in addition, equipped with a second passive joint having a second position sensor, wherein a second measured value is calculated from a chronological succession of position signals of the second position sensor and compared with the first threshold value and with the second threshold value when this has been defined,
wherein control of the movement of the instrument is interrupted when the first and the second measured value are smaller than the first threshold value, and
wherein the interruption of control is cancelled when either the first or the second measured value exceeds the first threshold value and the second threshold value when this has been defined, and/or if the control unit receives an activation signal that is independent of the movement.

12. A robotic surgery system, comprising at least one manipulator arm and an instrument held by the manipulator arm, an input unit for handling the at least one manipulator arm, with the input unit including a multi-linkage kinematic chain with active and/or passive joints and an open end, and a control unit, which converts movements of the input unit into movements of the manipulator arm and the instrument,
wherein the open end of the input unit is configured as a handpiece for controlling the movements of the instrument by an operator,
wherein the handpiece comprises at least a first passive joint and a second passive joint, and the first passive joint is equipped with a first sensor unit comprising a first position sensor, and the second passive joint is equipped with a second sensor unit comprising a second position sensor,
wherein the first sensor unit is adapted to transmit, in chronological succession, first position signals of the first position sensor to the control unit, and the second sensor unit is adapted to transmit, in chronological succession, second position signals of the second position sensor to the control unit,
wherein the control unit is adapted to calculate a first measured value from the chronological succession of the first position signals and a second measured value from the chronological succession of the second position signals, and
wherein the control unit is adapted to interrupt control of the movements of the instrument if the first measured value and the second measured value are smaller than a specified first threshold value.

13. The robotic surgery system as claimed in claim 12, wherein the first threshold value is specified to the effect that, in case the first and the second measured value are smaller than the first threshold value, the presence of a physiological tremor and, thus, an involuntary movement are ruled out.

14. The robotic surgery system as claimed in claim 12, comprising a fixation mechanism which, in case that the first and der second measured value are smaller than the threshold value, fix at least the active joints in their current positions.

15. The robotic surgery system as claimed in claim 14, comprising a timing circuit, which, in case the active joints are fixed, will cancel a coupling between the input unit and the at least one manipulator arm after a specified period of time has elapsed.

16. The robotic surgery system as claimed in claim 13, comprising a fixation mechanism which, when that the first and the second measured value are smaller than the threshold value, fix at least the active joints in their current positions.

17. The robotic surgery system as claimed in claim 16, comprising a timing circuit, which, when the active joints are fixed, will cancel a coupling between the input unit and the at least one manipulator arm after a specified period of time has elapsed.

18. The robotic surgery system as claimed in claim 12, wherein a second threshold value is defined that is greater than the first threshold value and is specified to the effect that the first or the second measured value being in excess of the second threshold value corresponds to the presence of a movement deliberately initiated by the operator.
